(19) United States
(12) Patent Application Publication
(10) Pub. No.: US 2008/0241925 A1
(43) Pub. Date: Oct. 2, 2008

(54) Three-Dimensional Self Assembly in Suspension of Adherent Cells

(76) Inventor: Angela Paz Judith Cruz, Brampton (CA)

Correspondence Address: HOWARD M COHN;PATENT ATTORNEY LLC, 21625 CHAGRIN BLVD #220, CLEVELAND, OH, 44122, US

(21) Appl. No.: 11/681,225

(22) PCT Filed: Sep. 2, 2005
(86) PCT No.: PCT/IB05/03785

§ 371 Date: Mar. 2, 2007

Related U.S. Application Data
(60) Provisional application No. 60/606,434, filed on Sep. 2, 2004.

(51) Int. Cl. C12N 5/08; C12M 1/00

(52) U.S. Cl. 435/383; 435/395; 435/289.1

(57) ABSTRACT

Methods and vessels for three-dimensional self-assembly of cells in suspension. The methods and vessels involve pre-coating a cell culture vessel with an adhesion-inhibiting substance such as Pluronic prior to culturing a cell therein.

This application claims the benefit of U.S. Provisional Application No. 60/606,434, filed on Sep. 2, 2004, which is incorporated herein by reference.

### FIELD OF THE INVENTION

The present invention relates to a method of promoting three-dimensional self-assembly in suspension of adherent cells. The present invention also relates to three-dimensional cellular assemblies produced by the inventive method and uses thereof.

### BACKGROUND OF THE INVENTION

Cells grow and function autonomously in two-dimensional culture, but require organization into tissues in order to create growth and function as required by the organism. Cell culture in two-dimensional plastic dishes has been found unable to support cell differentiation as measured by in vivo functions such as production of differentiation-specific hormones and other proteins. Rather, three-dimensional support is required for tissues to grow and function normally.

Current three-dimensional tissue culture methods successful in growing adherent cells require use of scaffold material such as Cytodex 3 or styrofoam beads. Adherent cell types, in particular, do not engage in self-assembly in the absence of scaffold support. Human mammary epithelial cells (HMEC), for example, will not self-assemble when placed directly into a bioreactor. As noted above, three-dimensional tissue-like constructs grown on scaffold materials often introduce the problem of how to retrieve single cells for subsequent cell assays.

Tissue-like cultures in terms of morphology and functionality are often complex and time-consuming to establish. Adherent cell types, in particular, do not engage in self-assembly in the absence of scaffold support. Human mammary epithelial cells (HMEC), for example, will not self-assemble when placed directly into a bioreactor.

Some researchers have employed the use of a viscous gel such as methylcellulose to support suspension cultures which give them a flexibility to perform cell-based growth assays as well as other calorimetric assays. However, methylcellulose cultures have generally been successful only with intrinsically non-adherent cell types such as hematopoietic cells. In my experiments, use of methylcellulose did produce suspension cultures, but cell growth was highly retarded, and there was no discernible organization conferred to the suspended cells. Thus, methylcellulose is not suitable for encouraging three-dimensional self-assembly in suspension of traditionally adherent cells.

Studies of cell growth and function to emulate tissue-like organization have been conducted involving embedding cells in agarose and dispersing cells in Matrigel strands, as the use of reconstituted extracellular matrix (ECM) for in vitro three-dimensional scaffolding is often reported to support tissue-like character. In addition, there have been efforts for improvement of the ECM material itself for cell culture, such as use of dimethylethylenediamine (DMEDA)-modified (methoxypolyethylene glycol) PEG-derivatized collagen to enhance attachment Tiller et al., Biotechnol Bioeng. 2001 May 5; 73(3):246-52. Alternate synthetic scaffolds made of polymers such as polylactide, polyglycolide, and polylactide-co-glycolide have also been widely employed in tissue engineering but have not consistently resulted in high degrees of success.

Current three-dimensional tissue culture methods successful in growing adherent cells require use of scaffold material such as Cytodex 3 or styrofoam beads. Tissue engineering in bioreactors often involves the use of commercially available collagen-coated dextran beads for cell seeding into three-dimensional constructs. Khaoustov et al., In Vitro Cell Dev Biol Anim. 1999 October; 35(9):501-9. However, three-dimensional tissue-like constructs grown on scaffold materials often introduces the problem of how to retrieve single cells thus limiting assayability, because cells grown in current scaffold materials are difficult to disaggregated. In view of the above, there is a long-felt, but unmet need for a method of promoting three-dimensional self-assembly in suspension of adherent cells, in particular.

### SUMMARY OF THE INVENTION

The present invention addresses the above-discussed need by providing methods and vessels useful for promoting three-dimensional self-assembly of cells in suspension. The invention is directed to such methods and vessels as well as to the cellular assemblies produced therewith. Examples of desirable aspects of self-assembly of a number of cell types include effective repulsion from the vessel surfaces, increased viscous support, and modified equilibria favoring cell-cell association. The present invention exploits the unexpected and surprising discovery that pre-coating a cell culture vessel with an adhesion-inhibiting substance, prior to culturing a cell in the vessel, encourages three-dimensional self-assembly in suspension of cells. In a preferred embodiment of the invention, the adhesion-inhibiting substance is a pluronic. In another preferred embodiment, the adhesion-inhibiting substance is a pluronic which has been dried onto an interior surface of the vessel. In another preferred embodiment, the cell is a human mammary epithelial cell which is a traditionally adherent cell. Other aspects of the present invention will become apparent to those skilled in the art from a study of the following description of the invention and non-limiting experimental results.

### DESCRIPTION OF THE DRAWINGS

FIG. 1 is a graph describing growth of adherent human mammary epithelial (designated WH612/3) cells. Absorbance readings at 260 nm show assessments of total DNA contents on the 3^{rd} and 6^{th} day of culture. Day 0 readings were for 100,000 cells initial seeding. Data comes from three replicate samples per time point.

FIGS. 2A-2B are micrographs of cellular assemblies (FIG. 2A) produced according to a method of the invention, disaggregated cells (FIG. 2B). FIG. 2A shows the progression of disaggregation with time of exposure to trypsin at 5 minutes. FIG. 2B shows progression of disaggregation at 10 minutes.

FIG. 3 is a cell cycle analysis of 10⁶ WH612/3 cells on the 8^{th} culture passage transfer (WH612/3p8), grown in surfactant (Pluronic F68)-coated versus uncoated 100 mm polystyrene dishes in MEGM medium at 5% CO₂/37° C. Column legends: −sa=WH612/3p8 cells plated at 10⁶ cells on 100 mm dishes and grown for 3 days without addition of surfactant; +sa=WH612/3p8 cells plated at 10⁶ cells on 100 mm dishes and grown for 3 days with surfactant. Row legends: micrographs=pictures taken at 200× magnification of the cultures on the 3^{rd} day prior to processing for cell cycle analysis; PI signal=distribution histograms of propidium iodide staining indicating DNA content in the cells; M1 fraction=percentage of the cell population in the G0/G1 phase of the cell cycle; M2 fraction=percentage of the cell population in the S phase of the cell cycle transitioning from G1 to G2; M3 fraction=percentage of cell population in the G2 phase of the cell cycle; M3 peak ch, M1 peak ch=peak channels for the G2 and G1 cell distributions; M3/M1 ratio=results of dividing M3 peak ch by the M1 peak ch which remain close to the expected number 2 indicating a doubling of DNA content in the cells. M1, M2, and M3 fractions (in percentage of cell population) showed comparable numbers between the two culture conditions: M1 values were 78.04 without surfactant and 80.23 with surfactant; M2 values were 2.31 without surfactant and 5.60 with surfactant; and M3 values were 12.63 without surfactant and 10.70 with surfactant. M3/M1 peak channel ratios were calculated to be 1.92 and 1.94, respectively.

FIGS. 4A-4B show self-assembly of spherical structures (FIG. 4A), and outgrowths (FIG. 4B). FIG. 4A shows cells grown pluronic culture for one day. FIG. 4B shows these cells of FIG. 4A after being replated onto untreated 2-dimensional culture dishes and grown for seven day period.

FIG. 5 shows results of an experiment done to compare whether the addition of surfactant to the culture medium or coating of surfactant to the vessel would be more effective in producing the suspended assemblies of normally adherent cells.

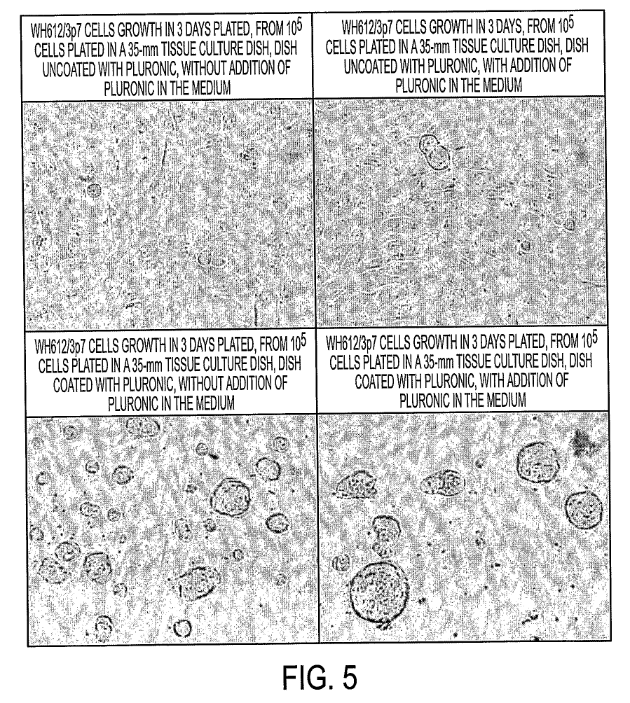

FIG. 6 shows WH612/3p8 cells cultured in Pluronic F68-coated 100 mm polystyrene dishes in MEGM medium at 5% CO₂/37° C. at 106 initial seeding. Pictures were taken in sections to complete the whole structure.

FIG. 7 shows WH612/3p8 cells cultured in Pluronic F68-coated Opticell Chambers in MEGM medium at 5% CO₂/37° C. at 106 count on initial seeding.

### DETAILED DESCRIPTION OF THE INVENTION

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full.

Any suitable materials and/or methods known to those of ordinary skill in the art can be utilized in carrying out the present invention; however, preferred materials and/or methods are described. Materials, reagents and the like to which reference is made in the following description and examples are obtainable from commercial sources, unless otherwise noted. Discussion of the examples provided herein, illustrate certain preferred embodiments and aspects of the present invention and are not to be construed as limiting the scope thereof.

Methods and vessels for culturing cells and organized cellular assemblies produced therewith are described here. More specifically, certain adhesion-inhibiting substances, when applied as coatings to cell culture vessels, prior to culturing cells therein, unexpectedly promote cell-cell interactions which in turn permit three-dimensional organized self-assembly of cells in suspension. Cellular assemblies produced according to the method and with vessels described here are amenable to disaggregation, for retrieval of single cells for subsequent cell assays.

Generally, according to the methods described here, an adhesion-inhibiting substance is delivered to and used to coat a cell culture vessel. Cells are detached, harvested, subsequently re-suspended and cultured in the coated vessel. Growing cells according to these methods results in organized cellular assemblies grown in suspension that may then continue to be grown in the same vessel, transferred to other vessels for further growth and differentiation, or disaggregated for subsequent assays.

### Cells

The methods and vessels described here are useful for growing both nonadherent and adherent cells. This is particularly useful for growing traditionally adherent cells, which do not engage in self-assembly in the absence of scaffold support. Adherent cells are traditionally grown on an adhesive substrate as a monolayer culture. The methods described here, however, allow adherent cells to be grown in suspension, and further to grow in an assembly. In a preferred embodiment, WH612/3 human mammary epithelial cells were used.

The origin of WH612/3 cells is described by Richmond et al., Abstract P8-24 at 2002 Era of Hope Department of Defense Breast Cancer Research Program Meeting (2002), which is incorporated herein by reference. Examples of other traditionally adherent cells aside from mammary epithelial cells useful in the methods described here include but are not limited to other human cells, animal cells, plant cells, and microbial cells.

### Vessel Coating

Essentially, any substance or combination of substances that provides a reasonably stable interaction with the material of a vessel surface, thereby preventing adhesion of cells to said surface and thereby further promoting cell-cell interaction, may be used as a vessel coating. For example, materials that provide sufficiently strong hydrophobic bonds or covalent complexes are highly desirable. Polymers expected to be useful as adhesion-inhibiting coating substances may contain both hydrophilic and hydrophobic moieties, consisting of at least two monomers, i.e., a hydrophilic monomer and a hydrophobic monomer, and more preferably from at least three monomers such as poloxamers which are block copolymers of ethylene oxide (EO) and propylene oxide (PO).

Certain cell-protective substances have been shown to protect freely suspended cells from agitation and aeration damage. Such substances include but are not limited to pluronic polyols, various derivatized celluloses and starches such as HES, protein mixtures, polyvinyl-pyrrolidones (PVP), polysaccharides such as dextrans and other sugars, polyethylene glycol (PEG), and polyvinyl alcohol and may also be used as vessel coating substances. Papoutsakis et al., Trends Biotechnol. Review 1991 September; 9(9):316-24 (hereinafter “Papoutsakis”), which is incorporated herein by reference. The use of these substances may be additionally advantageous as such substances may be nontoxic, nonimmunogenic, and/or biologically inert.

Further, surface tension-lowering agents such as detergents like bile salts and ionic surfactants like cholic acid are also found to promote cell suspension and may be used alone or in combination with other surfactants and nonsurfactant substances that are likewise determined to be useful as vessel coating substances. Notably, the greater the proportions of PEG (polyethylene glycol, polyethylene oxide, or polyoxyethylene), other non-tissue binding polymers, PLL (polylysine), and neutral polysaccharides comprising a copolymer, the greater the ability that copolymer may have in preventing cell adhesion. Hubbell et al., (2004) U.S. Pat. No. 6,743,521, which is herein incorporated by reference.

Other specific features of polymeric substances that may affect cell adhesion include the chemical nature of the tissue-binding and the non-binding domain, the mass and number ratios of binding to non-binding domains, presence of hydrolysis-susceptible sites, and the inclusion of sites with particular biological affinity. Additionally, the lengths of the polymeric materials which would result in prevention of adhesive interactions sufficient to allow cellular assemblages may be empirically determined through experimentation. Variations in desired biological performance may also be attributed to the degree of polymer-cell binding, cell-cell repulsion, duration of polymer-cell binding, duration of cell-cell repulsion, and loss of polymer-cell binding or cell-cell repulsion.

In a preferred embodiment, the vessel coating is a pluronic. In yet another preferred embodiment, the vessel coating is Pluronic F68. Pluronics and reverse pluronics are poly(oxyethylene)-poly(oxypropylene) block copolymer polyols of various molecular weights and percentages of the hydrophobe poly(oxypropylene). Pluronic is a triblock polymer, with a central polypropylene oxide block that adsorbs to hydrophobic surfaces such as polystyrene and flanking hydrophilic polyethylene oxide blocks. BASF Corporation is the source of pluronic (EO-PO-EO) and tetronic (PO-EO-PO) surfactants. Based on a procedure by Becher, polyols with the largest hydrophile-lipophile balance (HLB) values are found to correlate with a greater ability to protect animal cells. Murharnmer et al., Biotechnol Prog. 1990 March-April; 6(2):142-8. Fisher et al., (2001) U.S. Pat. No. 6,312,685 B1, which is herein incorporated by reference, discloses that Pluronic F68 inhibits cell aggregation by adsorbing onto the surface of the red blood cell. Accordingly, the hypothesis is put forward that the mechanism allowing for the phenomenon described here involves a deterrence of cell adhesion by the pluronic coating on the interior of the vessel, permitting the cells to associate with each other, although this tentative explanation is not meant to limit the scope of the invention. The pluronic may still be adsorbed onto the cell surface and other mechanisms which are unknown but which result in observed cell self-association may be in force. Larger pluronics such as F88, F98, F108, and F127 have greater hydrophobic segments and bind to red blood cells, making them self-associate. On the other hand, it is at least suggested that smaller pluronics, such as F38, and propionic acid, may cause cellular assembly in other ways.

Further, as noted above vessel coating substances may be used in combination. Combinations may allow for increased exploitation of various desired properties such as dry times, complexation, and the like. Wang et al., (2005) U.S. Pat. No. 6,838,078, which is herein incorporated by reference. In a preferred embodiment, polyalkoxylated, and in particular, polyethoxylated, nonionic surfactants are used in combination with pluronic as these surfactants are reportedly able to stabilize the film-forming property of pluronic, and certain like polymers.

The concentration of surfactant comprising coating substances will vary depending upon the nature of the vessel-coating substance interface. For example, if the coating substance is covalently bonded, a higher contact concentration can be achieved by virtue of the covalent coating. Accordingly, the concentration of surfactants may be higher than if a non-covalently bonded coating is used.

While the skilled artisan will understand that different applications require different vessel coatings and different concentrations of the coatings, in general solutions of an adhesion-inhibiting coating substance, such as Pluronic F68, may be applied to a culture vessel in a concentration of about 10 wt. %, about 9 wt. %, about 8 wt. %, about 7 wt. %, about 6 wt. %, about 5 wt. %, about 4 wt. %, about 3 wt. %, about 2 wt. %, about 1 wt. %, or less than 1 wt. %. In a preferred embodiment, a solution of about 0.1 wt. % to about 30 wt. % Pluronic F68 is applied to a surface of a culture vessel, more preferably about 1 wt. % to about 20 wt. % Pluronic F68, most preferably about 10 wt. % Pluronic F68.

### Preparation of Culture Vessels

### Carrier Vehicles for Coating Substance

The vessel coating substance may be dissolved or dispersed in a vehicle. Effective carrier vehicles include aqueous solvents, which are solutions consisting primarily of water, examples of which are pH buffers, organic and inorganic salts, alcohols, sugars, amino acids, or surfactants.

In a preferred embodiment, the vehicle is water, either essentially or substantially purified, particularly distilled water, deionized water, injectable-grade water, or the like, not excluding tap water or the like, containing low amounts of inorganic salt impurities, with or without additional substances that confer stability, uniformity, resistance, and durability to the coating in various environments such as pH, temperatures, humidity levels, viscosities, or conditions of processing, including repeated freeze-thaw and heat dissolution, over various durations of time.

Dispersion or dissolution of the coating substance may also be accomplished using organic solvent carrier vehicles such as inert alcohol solvent, nitrites, amides, esters, ketones, and ethers.

In a preferred embodiment, Pluronic F68 is dissolved in water to form a solution of from about 0.1 wt. % to about 30 wt. %, more preferably about 1 wt. % to about 20 wt. %, most preferably about 10 wt. %.

Delivery of the coating substance Delivery of the coating substance may be accomplished using traditional liquid transfer techniques. Delivery of the coating substance may be facilitated via agitation under controlled temperatures. Hellung-Larsen P. J. Biotechnol. 2005 Jan. 26; 115(2):167-77 (hereinafter “Hellung-Larsen”), which is hereinafter incorporated by reference. In a preferred embodiment, Pluronic F68 dispersed in water was agitated at 25° C. and transferred via micropipeffe.

### Removal of Carrier Vehicles

Following the application of coating material, carrier vehicles either may be removed by air-drying or other methods such as washing out with water or saline. Coating substances also may be purified by precipitation or oxidation. Drying time is preferably overnight, but also may be done anywhere in the range of 1 hour and up, preferably 2-48 hours, more preferably 2-24 hours, more preferably, 2-12 hours, more preferably 4-10 hours, still more preferably 6-8 hours. Depending upon the vessel material and coating substance used, the melting and degradation points of which may readily be determined by one skilled in the art, drying may take place at a temperature of about 250° C., about 200° C., about 150° C., about 100° C., about 50° C., about 25° C., about 20° C., or about 15° C. In a preferred embodiment, carrier vehicles were removed by air drying for a duration of 12 hours at 20° C.

In a preferred embodiment, a solution of Pluronic F68 is dissolved in water, at a concentration of about 1 wt. % to about 20 wt. %, more preferably about 10 wt. %, and is added to a polystyrene tissue culture dish. The vessel is then rotated to distribute the solution of Pluronic F68 over a surface of the vessel. The Pluronic F68 coating is allowed to air-dry overnight.

In a further preferred embodiment, a solution of Pluronic F68 is dissolved in water, at a concentration of about 1 wt. % to about 20 wt. %, more preferably about 10 wt. %, and is added to an Opticell chamber. The vessel is then rotated to distribute the solution of Pluronic F68 over the interior surface of the chamber, and then withdrawn using a syringe. The Pluronic F68 coating is allowed to air-dry overnight.

### Stabilization of Vessel Coating

Either concurrently or following delivery of the coating substance and removal of carrier vehicles, stabilization of the coating via physical and/or chemical methods is highly desirable. Examples of physical methods for stabilizing the coating to an extent sufficient to allow assemblage of cells include but are not limited to blow-drying, freeze-drying, and other drying techniques, evaporation or boiling, heat vaporization, vacuum deposition, vapor deposition, salt deposition or crystallization, sol-gel phase shifting, low-temperature solidification, pumping, spraying, misting, atomization, micronization, microfluidics, photolithography, contact-transfers, mask printing, casting, molding, painting, adsorption, thermal bonding, microwave cross-linking, ultrasonic bonding, laser bonding and molecular beam deposition. Examples of chemical methods for stabilizing vessel coatings include but are not limited to surface charging, ionic charging, electrostatic bonding, electrovalent bonding, chemical bonding, covalent binding, electrolysis, and polymerization such as free radical polymerization, solution or ethanol polymerization, and emulsion polymerization.

Further, resistance to removal of the coating during actual use or contact with intended biological materials and retention of desired biological effects are preferred. Resistance to removal of the coating during actual use or contact with intended biological materials and retention of desired biological effects are preferred. Zamora et al teach that utilizing sterile techniques in preparation of the coated dishes is preferred; otherwise it may be possible to purify the coating by precipitation or oxidation, and ensuing products may be sterilized, for example, by gamma radiation, as an option. Zamora et al., (2005) U.S. Pat. No. 6,921,811, which is herein incorporated by reference.

### Media Formulations

Cell culture media useful herein refers to any medium in which cells are maintained in vitro in an active and viable state. A useful media formulation may include carbohydrates, proteins, amino acids, lipids, vitamins, minerals, salts, buffers, trace elements, and various other supplements such as an alcohol, a sterol, or a soluble carboxylic acid, as well as blood (serum) and/or tissue (pituitary) extracts. Bertheussen, (2004) U.S. Pat. No. 6,833,271 B2 (hereinafter “Bertheussen”), which is herein incorporated by reference.

In a preferred embodiment, the medium, specifically suited for culturing human mammary epithelial cells, is Mammary Epithelial Basal Medium (MEBM) from Cambrex, supplemented with 0.4% v/v bovine pituitary extract (BPE), 5 μg/ml bovine insulin, 0.5 μg/ml hydrocortisone, 3 ng/ml human epidermal growth factor, 50 U penicillin, and 50 μg of streptomycin. Together this media is referred to herein as MEGM Mammary Epithelial Growth Medium).

Additionally, surfactants discussed above that may comprise the coating substance may likewise be added to the culture media. More particularly, some non-ionic surfactants such as those of the polyoxyethylene sorbitan monooleate type, like Tween 80, are known not to interfere with the action of the cell culture medium, and are sometimes added. Similarly, as discussed with regard to effectiveness as coating substances, those substances known to protect freely suspended cells from agitation and aeration damage may be added to cell culture media. Again, such substances include but are not limited to pluronic polyols, various derivatized celluloses and starches such as HES, protein mixtures, polyvinyl-pyrrolidones (PVP), polysaccharides such as dextrans and other sugars, polyethylene glycol (PEG), and polyvinyl alcohol. Papoutsakis. The use of these substances may be additionally advantageous in culture media by virtue of their demonstrated nontoxicity, nonimmunogenicity, and/or biological inertia.

Pluronic F68, in particular, has been shown to have desirable cell-protective properties Xu et al., Chin J. Biotechnol. 1995; 11(2):101-7, which is herein incorporated by reference. Pluronic F68 is reported not to induce morphologic alteration of cells (Bregman et al., Fundam Appl Toxicol. 1987 July; 9(1):90-109, which is herein incorporated by reference) nor bind to cells with any significant affinity. Hellung-Larsen. Further, as disclosed in by Bertheussen and as noted above, Pluronic F68 is found to provide mechanical protection to cells. PVP-10 similarly provides mechanical protection to cells while PEG and propylene glycol improve cell viability in culture, and cholic acid aids in cell growth. Accordingly in a preferred embodiment, these additives are present in culture media. In a preferred embodiment, Pluronic F68 is added to the culture media at a final concentration of about 0.01 wt. % to about 25 wt. %, more preferably about 0.1 wt. % to about 10 wt. %, most preferably about 1 wt. %.

Also as discussed above with respect to coating substances, surface tension-lowering agents such as detergents like bile salts and ionic surfactants like cholic acid are found to promote cell suspension and thus may also be useful additives for media formulations.

Further, after structures have formed in culture, adhesive molecules such as extracellular matrix, for example, fibronectin and its derivations, peptide mimcs of fibronectin, laminin, vitronectin, thrombospondin, gelatin, collagen and its subtypes, gelatin, polylysine, polyornithine, and other adhesive molecules or derivatives or mimics of other adhesive molecules and the like, as disclosed in Zamora et al., (2005) U.S. Pat. No. 6,921,811, may also be added to the culture. In a preferred embodiment, human collagen IV is added to a concentration of 10⁻⁸ g/ml for conducting differentiation studies and for tissue engineering.

The addition of growth factor molecules such as insulin-like growth factors, and molecules such as chemokines, drugs such as antibiotics and anti-cancer medications, and hormones such as insulin, estrogen, progesterone, oxytocin, prolactin, and human placental lactogen is considered useful for inducing growth in human mammary epithelial cells. Other factors may be suitable for other cell types such as other epithelial cells, muscle cells, nerve cells, and connective tissue cells and are likewise contemplated.

Organized cells resulting from culturing cells according to the methods herein may be useful for continuous growth in the same vessel or transfer to other vessels such as the NASA bioreactor. In a preferred embodiment, the WH612/3 cells are grown in Pluronic F68-coated dishes for 3 days then transferred and cultured in the NASA bioreactor. It was observed that the structures from such cultures become more compact with the passage of the time period of observation of up to 10 days in the NASA bioreactor. In a control study, WH612/3 cell placed directly into a bioreactor did not self-assemble.

### Vessels

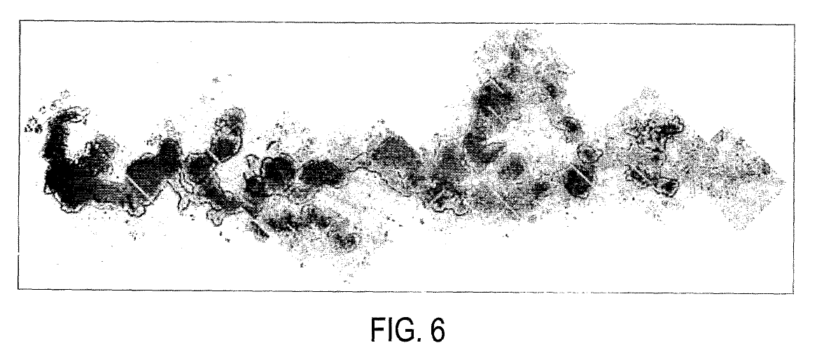

Any vessel suitable for containing biological materials, such as those used for cell culture, including, but not limited to, culture dishes, culture flasks, (multi)well plates, culturing membranes, Opticell chambers, culture bottles, suspension culture systems, bioreactor culture systems, fermentation culture systems, perfusion culture systems, and others know to the skilled artisan, may be used in the methods disclosed herein. Lee et al., (2005) U.S. Pat. No. 6,900,056, which is herein incorporated by reference. The skilled artisan will understand that suitable vessels may be constructed of a variety of materials, including, but not limited to, (methylated) glass, silicone (rubber), polystyrene, and polylactic-co-glycolic acid. In a preferred embodiment, polystyrene culture dishes are used. In yet another preferred embodiment, Opticell Chambers are used. As shown in FIG. 6, growth of 10⁶ total cells at initial seeding in polystyrene dishes resulted in the formation of an extensive gland-like assembly after one day of culture. Growth of 10⁶ total cells at initial seeding in an Opticell Chamber resulted in the formation of a similar gland-like assembly.

Other types of surfaces such as macrocapsular surfaces such as ultrafiltration and hemodialysis membranes, non-microencapsulated hollow fibers for immunoisolation of tissue, and fullerenes/buckyballs may be used. Other configurations include those shaped relative to an internal or external supporting structure as well as microfabricated and nanofabricated shapes. Zamora et al., (2005) U.S. Pat. No. 6,921,811.

While the skilled artisan will understand that various culture conditions may be used, based on the identify of the cells being cultured, in a preferred embodiment, WH61213 cells are cultured in MEGM supplemented with 0.4% v/v bovine pituitary extract (BPE), 5 μg/ml bovine insulin, 0.5 μg/ml hydrocortisone, 3 mg/ml human epidermal growth factor, 50 U penicillin, and 50 μg of streptomycin. The vessels are cultured at about 37° C., under a humidified atmosphere of about 5% CO₂.

### Applications and Uses

### Storage

Cellular assemblies produced by the inventive method may be amenable to cryostorage freezing, low-temperature retrieval, and/or post-thaw recovery of cells.

### Studies of Apoptosis and Anti-Apoptosis

Due to increased cellular protection conferred by the cell-protective properties of coating substances used here, cellular assemblies produced by these methods may be useful for production of proteins or expression of cellular markers involved in the cellular processes of apoptosis and anti-apoptosis.

### Single Cell Assays

Three-dimensional cellular assemblies produced using the methods described here are amenable to disaggregation for subsequent single-cell assays. Such assays may include single cell counts, colony counts, viability assays, growth assays, DNA assays, and gene and protein expression assays. Methods of harvesting/detachment include but are not limited to enzymatic or proteolytic methods such as trypsinization and ionic manipulation such as chelation.

### Differentiation Studies

Cellular assemblies produced by the inventive method may be useful for production of proteins and expression of cellular markers involved in the cellular processes of differentiation and dedifferentiation. Further, the inventive method provides for more direct comparisons of cell behavior and protein expression among cells cultured in traditional two-dimensional monolayers in two-dimensional culture vessels, three-dimensional assemblies formed in two-dimensional culture vessels, three-dimensional assemblies in rotating vessels such as the NASA bioreactor, three-dimensional assemblies in Opticell chambers, and other derivatives.

These assays can serve to evaluate cell differentiation that may lead to tissue-equivalence and technologies used for tissue engineering including applications involving cellular substratum such as the extracellular matrix, as these organized structures have been demonstrated to have ability to accept extracellular matrix such as collagen IV, laminin, and fibronectin, or other adhesive molecules. In a preferred embodiment, human collagen IV was added into the growth medium containing structures that have already been formed using the methods herein to a working concentration of 10⁻⁸ g/ml. The addition of collagen IV extracellular matrix did not visibly disrupt the progression of the structures. As cells are typically in contact with extracellular matrix in vivo and tissue engineering is largely directed to emulating in vivo conditions in vitro, the addition of collagen IV and the like is considered advantageous.

The products disclosed herein may find medical application in artificial blood vessels, blood shunts, nerve-growth guides, artificial heart valves, prosthetics, cardiovascular grafts, bone replacements, wound healing, cartilage replacement, urinary tract replacements, and the like, for conditions such as burns, cardiovascular ischemia, peripheral vascular ischemia, vascular aneurysms, bone fractures, skeletal defects, orthopedic trauma, cartilage damage, cancer treatment, antibacterial treatment, neural damage, myocardial infarction, peripheral vascular occlusion, ocular degeneration, kidney ischemia, and the like. Zamora et al., (2005) U.S. Pat. No. 6,921,811

### SPECIFIC EXAMPLES

The following examples utilize a common set of procedures, using of the same cell stock, and having the same requirements. Unless otherwise stated herein, the following procedures and conditions were used in each of the forgoing examples.

The cell stock used for these studies was WH612/3, produced by centrifuging collected cells from the fifth passage transfer (p5) at ˜1,000 rpm, for 5 minutes, prior to addition of 10% dimethyl sulfoxide (DMSO) in growth medium, and freezing in liquid nitrogen at 1 million cells/ml per cryovial.

Cryovials containing the cells were retrieved as needed from storage in the nitrogen (N₂) tanks, placed in a 37° C. water bath for a few minutes until the supernatant had thawed. The cells in the bottom of the cryovial were then collected using a Pasteur pipette and distributed onto 2 100-mm tissue culture dishes containing 10 ml MEGM each (Mammary Epithelial Basal Medium (MEBM) from Cambrex, supplemented with 0.4% v/v bovine pituitary extract (BPE), 5 μg/ml bovine insulin, 0.5 μg/ml hydrocortisone, 3 ng/ml human epidermal growth factor, 50 U/ml penicillin, and 50 μg/ml of streptomycin, referred to as MEGM (for Mammary Epithelial Growth Medium) herein). The dishes were then placed in 5% CO₂/37° C./humidified incubator overnight. On the first day after cell retrieval, the medium containing some DMSO from the freezing procedure was replaced with 10 ml fresh medium to each plate.

Cells were kept inside 5% CO₂/37° C./humidified incubators. Refeeding, or medium changes, were done every 2-3 days, which consists of discarding old growth medium and replacing with fresh medium.

The passage notation refers to the number of times the cells were transferred into another dish prior to confluence since being obtained from the tissue specimen, with each passage lasting about 1 week. Each passage transfer consisted of observation that the cells in the dish were still growing actively or exponentially at a subconfluent density and harvested from the dish by the usual trypsinization procedure as follows. Cells attached to the dish were rinsed with 5 ml phosphate-buffered saline (PBS), after which they were trypsinized using 1 ml 0.01% trypsin/0.02% EDTA (ethylenediaminetetraacetic acid) in PBS, incubated at 37° C., and observed at 5 minutes under a light microscope to check when cells were lifted off from the dish. The detached cells were then pipetted up and transferred into a 15 ml centrifuge tube with 1 ml 0.25 mg/ml soybean trypsin inhibitor.

The centrifuge tube containing the cells was swirled gently in order to ensure uniform distribution of the cells in the suspension prior to taking two volumes of 10 ul each of the cells suspension for counting in a hemacytometer. Each 10 ul volume was delivered to each hemacytometer chamber using a micropipette and counted. The average of counts of cells within the big squares of the hemacytometer were taken and multiplied by 10,000 in order to obtain the number of cells per ml. After the cell counts had been calculated, the tubes containing the cells were centrifuged at 1,000 rpm for 10 min at room temperature (˜25° C.) and resuspended to the desired number of cells/ml need for the experiment or further passage using MEGM.

Pluronic F68-coated tissue culture vessels were prepared by dissolving Pluronic F68 in water to form a 10 wt. % solution. An amount of this solution sufficient to coat a surface of the vessel, such as 0.1 ml for 35 mm tissue culture dishes, and 1 ml for 100 mm tissue culture dishes, was then added to the culture vessel, the vessel was rotated to fully cover a surface of the vessel, and then allowed to air dry overnight.

### Example 1

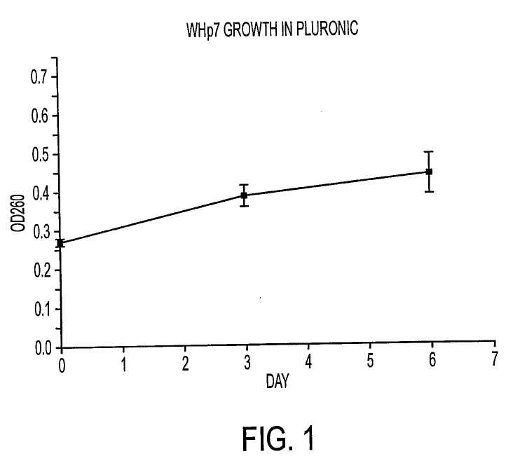

WH612/3p6 cells were trypsinized from untreated tissue culture dishes and counted using the hemacytometer. Cells were resuspended in MEGM to a concentration of 100,000 cells/ml and 1 ml each of the cell suspension were transferred to Pluronic F68-coated 35 mm dishes containing 1 ml MEGM each for growth at p7. Cells were allowed to grow inside a 5% CO₂/37° C. humidified incubator, prior to subjecting them to assay procedure at the different experimental time points. The cellular assemblies from each dish were collected into 5 ml centrifuge tubes and centrifuged at 1,000 rpm for 10 minutes at room temperature. Media were removed from the cells in each of the tubes, and the cellular assemblies were resuspended in 5 ml PBS wash and centrifuged again at 1,000 rpm for 10 minutes at room temperature. PBS was removed from the cells in each of the tubes prior to lysing the cells in each individual tube using 1 ml 0.1 N sodium hydroxide (NaOH). The cell lysates were recentrifuged at 1,000 rpm for 10 minutes at room temperature, and the resultant supernatants were then transferred into 1 ml cuvettes. Absorbances of the supernatants were read at 260 nm on the UV spectrophotometer. Time points in this experiment are the 3^{rd} and 6^{th} day of culture, as well as the day of plating itself (designated day zero). Day 0 readings were taken from three 1 ml volumes of 100,000 cells each after the cell counts. The three day zero samples were treated in essentially the same manner as the 3^{rd} and 6^{th} day samples, with stepwise removal of media, PBS washing, and NaOH lysing with the aid of centrifugation. Data comes from three replicate samples per time point. Growth was confirmed by increases in total DNA absorbances after 3 days and 6 days in pluronic culture. See FIG. 1.

### Example 2

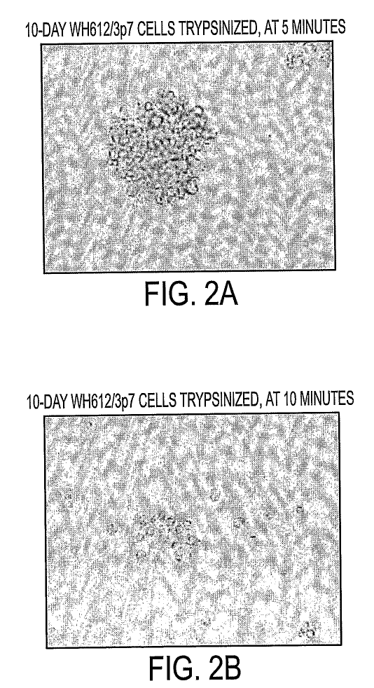

WH612/3p6 cells were trypsinized from untreated tissue culture dishes and counted using the hemacytometer. Cells were resuspended in MEGM to a concentration of 100,000 cells/ml and 1 ml each of the cell suspension were transferred to Pluronic F68-coated 35 mm dishes containing 1 ml MEGM each for growth at p7. Cells were allowed to grow inside a 5% CO₂/37° C. humidified incubator for 10 days prior to trypsinization. Cellular assemblies from each dish on the 10^{th} day of culture were collected into 5 ml centrifuge tubes and centrifuged at 1,000 rpm for 10 minutes at room temperature. Media were removed from the cells in each of the tubes, and the cellular assemblies were resuspended in 5 ml PBS wash and centrifuged again at 1,000 rpm for 10 minutes at room temperature. PBS was removed from the cells in each of the tubes prior to adding 1 ml trypsin solution. The tubes were incubated at 37° C. and cells were removed from the tubes for viewing under the microscope at 5 minutes and 10 minutes. Photomicrographs were taken using the 40× objective of the Leitz Fluovert microscope and a SPOT digital cooled-CCD camera. The micrographs shown in FIG. 2A shows the progression of disaggregation with time of exposure to trypsin at 5 minutes. FIG. 2B shows progression of disaggregation at 10 minutes. By the end of 10 minutes, disaggregation is seen to be complete.

### Example 3

WH612/3p6 cells were trypsinized from untreated tissue culture dishes and allowed to grow inside a 5% CO₂/37° C./humidified incubator for about 1 week to reach near-confluence, prior to being trypsinized and subcultured 1:2 into the next passage transfer (p7). WH612/3p7 cells were allowed to grow again inside a 5% CO₂/37° C./humidified incubator for about 1 week prior to being trypsinized and counted using the hemacytometer. Cells were resuspended in MEGM to a concentration of 1,000,000 cells/ml and 1 ml each of the cell suspension were transferred to both Pluronic F68-coated and uncoated 100 mm dishes containing 9 ml MEGM each for growth at p8. These WH612/3p8 cells were allowed to grow inside a 5% CO₂/37° C. humidified incubator for 3 days.

Photomicrographs were taken on the 3^{rd} day using the 40× objective of the Leitz Fluovert microscope and a SPOT digital cooled-CCD camera, prior to trypsinization. The cells in adherent cultures were trypsinized according to the general standard protocol discussed above, while cellular assemblies from the pluronic cultures were collected into 15 ml centrifuge tubes and centrifuged at 1,000 rpm for 10 minutes at room temperature. Media were removed from the cells in each of the tubes, and the cellular assemblies were resuspended in 10 ml PBS wash and centrifuged again at 1,000 rpm for 5 minutes at room temperature. PBS was removed from the cells in each of the tubes prior to adding 1 ml trypsin solution. Upon complete disaggregation of the assemblies in about 10 minutes, 1 ml soybean trypsin inhibitor was added. Cells from both the pluronic and non-pluronic cultures were transferred to a new 15 ml tube and centrifuged at 1,000 rpm for 5 min at room temperature. The supernatant was removed from each tube and replaced with 10 ml PBS. The tubes were centrifuged again at 1,000 rpm for 5 min at room temperature, before cells were resuspended with 1 ml PBS. 4 ml of cold 70% ethanol was added dropwise to the tubes while vortexing. Cells were incubated in the final mixture for 30 minutes at room temperature, after which the tubes are recentrifuged at 1,000 rpm for 5 min. The resultant supernatant is removed and replaced with 10 ml PBS. Cells were counted using the hemacytometer, and volumes corresponding to a concentration of 1,000,000 cells were transferred into new 5 ml tubes for staining. Tubes containing 1,000,000 cells each were centrifuged at 1,000 rpm for 5 min, and the supernatant was removed from the cells. Cells were resuspended in 0.1 ml PBS before the addition of 0.9 ml PI/RNase staining solution (50 ug PI:100 ug RNase type I-A in PBS). Samples were analyzed at 630 nm using the Becton Dickinson FACSCalibur flow cytometer and CellQuest flow cytometry data software program.

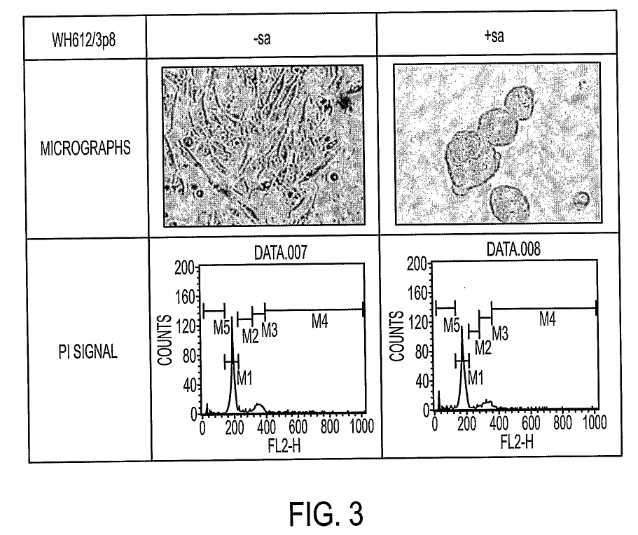

Micrographs and results are shown in FIG. 3. Column legends: −sa=WH612/3p8 cells plated at 10⁶ cells on 100 mm dishes and grown for 3 days without addition of surfactant; +sa=WH612/3p8 cells plated at 10⁶ cells on 100 mm dishes and grown for 3 days with surfactant. Row legends: micrographs=pictures taken at 200× magnification of the cultures on the 3^{rd} day prior to processing for cell cycle analysis; PI signal=distribution histograms of propidium iodide staining indicating DNA content in the cells; M1 fraction=percentage of the cell population in the G0/G1 phase of the cell cycle; M2 fraction=percentage of the cell population in the S phase of the cell cycle transitioning from G1 to G2; M3 fraction=percentage of cell population in the G2 phase of the cell cycle; M3 peak ch, M1 peak ch=peak channels for the G2 and G1 cell distributions; M3/M1 ratio=results of dividing M3 peak ch by the M1 peak ch which remain close to the expected number 2 indicating a doubling of DNA content in the cells. M1, M2, and M3 fractions (in percentage of cell population) showed comparable numbers between the two culture conditions: M1 values were 78.04 without surfactant and 80.23 with surfactant; M2 values were 2.31 without surfactant and 5.60 with surfactant; and M3 values were 12.63 without surfactant and 10.70 with surfactant. M3/M1 peak channel ratios were calculated to be 1.92 and 1.94, respectively.

The cell cycle distributions of the cells cultured in the two types of conditions were found to be comparable.

### Example 4

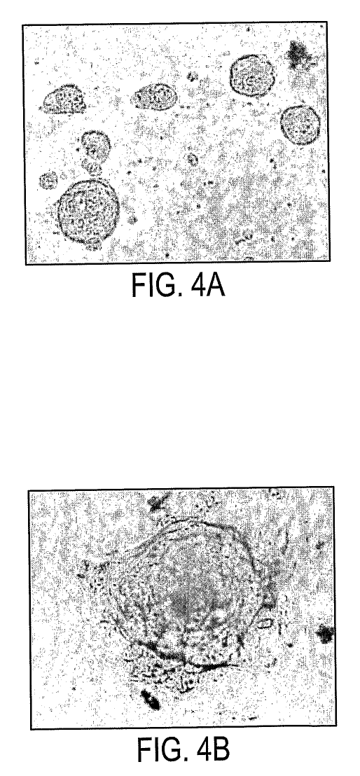

Actively growing WH612/3p6 cells were trypsinized from untreated tissue culture dishes and counted using the hemacytometer. Cells were resuspended in MEGM to a concentration of 100,000 cells/ml and 1 ml each of the cell suspension were transferred to Pluronic F68-coated 35 mm dishes containing 1 ml MEGM each for growth at p7. The assemblies were maintained inside a 5% CO₂/37° C./humidified incubator for 3 days, then transplanted onto uncoated 35 mm dishes on the 3^{rd} day (p8). Assemblies were allowed to grow inside a 5% CO₂/37° C./humidified incubator for 7 days. Micrograph in FIG. 4A shows assemblies that were formed in the coated dish by the first day, while micrograph in FIG. 4B shows subsequent growth of the assemblies in uncoated dishes on the 7^{th} day of transfer. Photomicrographs were taken on the 3^{rd} day using the 40× objective of the Leitz Fluovert microscope and a SPOT digital cooled-CCD camera. The cells as seen shown FIG. 4B reattached to the bottom of the dish (showing themselves to be viable) and produced outgrowths, thus showing themselves to be capable of further growth.

### Example 5

Actively growing WH612/3p6 cells were trypsinized from untreated tissue culture dishes and counted using the hemacytometer. Cells were resuspended in MEGM to a concentration of 100,000 cells/ml and 1 ml each of the cell suspension were transferred to both Pluronic F68-coated and uncoated 35 mm dishes containing 1 ml MEGM with or without the addition of 1% w/v Pluronic F68 each for growth at p7. The cells and cellular assemblies were maintained inside a 5% CO₂/37° C./humidified incubator for 3 days. Photomicrographs were taken on the 3^{rd} day using the 40× objective of the Leitz Fluovert microscope and a SPOT digital cooled-CCD camera.

Micrographs in the first row of the figure show the mode of cell growth in untreated dishes, while the micrographs of the bottom row show the mode of growth with Pluronic F68-coated dishes. The left column micrographs represent media without addition of Pluronic F68, while the micrographs of the right column represent media in which Pluronic has been added to a final concentration of 1%. Cells used were WH612/3p7 cells cultured in Pluronic F68-coated 35 mm polystyrene dishes in MEGM medium at 5% CO₂/37° C. at 10⁵ initial seeding. Using this matrix, it can be seen that coating is the more reliable method for producing the cellular assemblies in culture. The mere addition of pluronic to the medium also seems to confer a growth advantage to the cells in both the coated and uncoated conditions.

### Example 6

WH612/3p6 cells were trypsinized from untreated tissue culture dishes and allowed to grow inside a 5% CO₂/37° C./humidified incubator for about 1 week to reach near-confluence, prior to being trypsinized and subcultured 1:2 into the next passage transfer (p7). WH612/3p7 cells were allowed to grow again inside a 5% CO₂/37° C./humidified incubator for about 1 week prior to being trypsinized and counted using the hemacytometer. Cells were resuspended in MEGM to a concentration of 1,000,000 cells/ml and 1 ml each of the cell suspension were transferred to Pluronic F68-coated 100 mm dishes containing 9 ml MEGM each for growth at p8. These WH612/3p8 cells were placed inside a 5% CO₂/37° C./humidified incubator, and photomicrographs were taken in sections on the next day using the 40× objective of the Leitz Fluovert microscope and a SPOT digital cooled-CCD camera. It can be seen in FIG. 6 that an extensive gland-like assembly formed in one day of pluronic culture.

### Example 7

10 ml 1% w/v Pluronic F68 was placed in 100 mm tissue culture dishes or injected into Opticell chambers and kept in a running tissue culture hood at room temperature for 2 days. At the end of 2 days the Pluronic solution is either removed or syringed out and allowed to dry for 1 day, after which the culture vessels are ready for inoculation with cells.

WH612/3p6 cells were trypsinized from untreated tissue culture dishes and allowed to grow inside a 5% CO₂/37° C./humidified incubator for about 1 week to reach near-confluence, prior to being trypsinized and subcultured 1:2 into the next passage transfer (p7). WH612/3p7 cells were allowed to grow again inside a 5% CO₂/37° C./humidified incubator for about 1 week prior to being trypsinized and counted using the hemacytometer. Cells were resuspended in MEGM to a concentration of 1,000,000 cells/ml and 1 ml each of the cell suspension were transferred to Pluronic F68-coated Opticell chambers containing 9 ml MEGM each for growth at p8. These WH612/3p8 cells were placed inside a 5% CO₂/37° C./humidified incubator, and photomicrographs were taken on the next day using the 40× objective of the Leitz Fluovert microscope and a SPOT digital cooled-CCD camera.

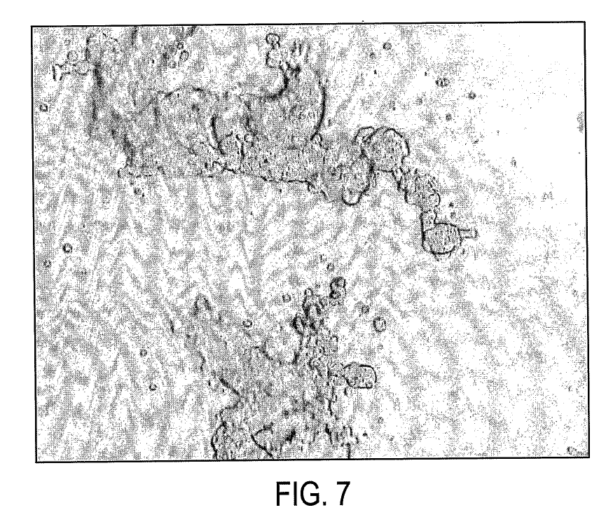

On the first day after plating, 1 ml of the medium in the Opticell chamber containing the structures was withdrawn and replaced with 1 ml 10⁻⁷ mg/ml human collagen IV was injected into the Opticell chambers containing the structures to a final concentration of 10⁻⁸ g/ml. The culture was maintained with no visible effects to the structures for 7 days. A gland-like structure formed following one day (see FIG. 7). Addition of extracellular matrix to a final concentration of 10⁻² mg/ml to the growth medium after such assemblies formed did not produce visible effects to the structures in subsequent days up to day seven.

While the invention has been described in detail and with reference to specific embodiments thereof, it will be apparent to one skilled in the art that various changes and modifications can be made therein without departing from the spirit and scope thereof.

1-22. (canceled)

23. A process for modifying an interior surface of one or more cell culture vessels prior to culturing cells in the one or more cell culture vessels, wherein the cells preferentially attach intercellularly over attachment to the substrate.

24. The process according to claim 23 wherein the preferential intercellular attachment between cells results in the formation of tissue-like or otherwise organized structures.

25. The process according to claim 24 wherein said organized structures are able to be replated.

26. The process according to claim 24 wherein said organized structures are able to be disaggregated for replating or cell-based assays.

27. The process according to claim 25 wherein said organized structures or the cells disaggregated from said organized structures are able to be replated for survival and/or growth.

28. The process according to claim 26 wherein said organized structures or the cells disaggregated from said organized structures are able to be replated for survival and/or growth.

29. The process according to claim 24, wherein said organized structures exhibit survival and/or growth.

30. The process according to claim 25, wherein said replated structures exhibit survival and/or growth.

31. The process according to claim 26, wherein said replated cells exhibit survival and/or growth.

32. A cell culture vessel having an interior surface pre-coated with a surface-active agent prior to culturing a cell in the cell culture vessel, and having the property of promoting intercellular attachment over attachment to the substrate, the cell culture vessel constructed by the steps of:
mixing a coating substance of an adhesion-inhibiting substance with a carrier vehicle;
delivering the coating substance and the carrier vehicle onto the interior surface of the cell culture vessel whereby said interior surface is coated with the adhesion-inhibiting substance and the carrier vehicle; and
removing the carrier vehicle from the cell culture vessel whereby the adhesion-inhibiting substance remains on the interior surface of the cell culture vessel.

33. The cell culture vessel according to claim 32 wherein the adhesion-inhibiting substance remaining on the interior surface of the cell culture vessel comprises a surfactant dried onto the interior surface of the vessel.

34. The cell culture vessel according to claim 32 wherein the coating substance is a pluronic.

35. The cell culture vessel according to claim 32 wherein the adhesion-inhibiting substance is stabilized onto the surface of the cell culture vessel.

36. The cell culture vessel according to claim 32 wherein said cell culture vessel with adhesion-inhibiting substance on its interior surface is sterilized.

37. A process for culturing traditionally adherent cells, comprising the steps of:
introducing cell culture media into a cell culture vessel having the property of promoting intercellular attachment over attachment to the interior surface;
introducing traditionally adherent cells into said cell culture vessel; and
culturing the traditionally adherent cells in the cell culture vessel, wherein the traditionally adherent cells preferentially attach intercellularly over attachment to the substrate.

38. The process according to claim 37, wherein the traditionally adherent cells preferentially self-assemble into 3-dimensional structures in suspension.

39. The process according to claim 37 including the step of introducing the cell culture medium with a surfactant.

40. The process according to claim 37, wherein scaffolding material is introduced to the cell culture medium.

41. The process according to claim 40, wherein the scaffolding material is an extracellular matrix.

42. A suspension culture of traditionally adherent cells, wherein said cells self-assemble into 3-dimensional structures in said suspension culture.

43. The suspension culture according to claim 42 wherein said traditionally adherent cells are human mammary epithelial cells.
